(19) United States
(12) Patent Application Publication
(10) Pub. No.: US 2024/0281974 A1
(43) Pub. Date: Aug. 22, 2024

(54) INTRAORAL IMAGE PROCESSING DEVICE AND INTRAORAL IMAGE PROCESSING METHOD

(71) Applicants: MEDIT CORP., Seoul (KR); MEDIT CORP., Seoul (KR)

(72) Inventors: Dusu KIM, Seoul (KR); Hotaik LEE, Seoul (KR)

(73) Assignees: MEDIT CORP., Seoul (KR); MEDIT CORP., Seoul (KR)

(21) Appl. No.: 18/569,357

(22) PCT Filed: Jun. 22, 2022
(86) PCT No.: PCT/KR2022/008832

§ 371 Date: Dec. 12, 2023

(30) Foreign Application Priority Data
Jun. 25, 2021 (KR) 10-2021-0083183
Dec. 16, 2021 (KR) 10-2021-0180849

(51) Int. Cl. G06T 7/00

(52) U.S. Cl. CPC G06T 7/0014; G06T 2207/30036

(57) ABSTRACT

Disclosed embodiments relate to an intraoral image processing method and an intraoral image processing device, wherein the intraoral image processing method according to an embodiment may include acquiring three-dimensional intraoral data by scanning an oral cavity including teeth, acquiring a tooth region including the teeth in the three-dimensional intraoral data, acquiring, based on the tooth region, a gingival region having an edge formed as a curve having a smaller curvature than a curvature of an edge of the tooth region, and displaying the tooth region and the gingival region as a final selection region.

### TECHNICAL FIELD

Disclosed embodiments relate to an intraoral image processing device and an intraoral image processing method, and more particularly, to an intraoral image processing device and an intraoral image processing method for determining a selection region to make a gingival region selected from three-dimensional scan data look natural when the gingival region is expanded.

### BACKGROUND ART

Recently, to obtain intraoral data of patients, a method of acquiring intraoral images by inserting a three-dimensional scanner into the oral cavity of a patient has been used. By scanning a patient's oral cavity using a three-dimensional scanner, three-dimensional data about an object such as the patient's teeth, gums, and jawbones may be acquired, and the acquired three-dimensional data may be used for the treatment or correction of patient's teeth.

To view the simulation results after scanning the patient's teeth and gingiva, it is necessary to expand the gingiva into a good-looking shape because simulation results may not look good when only the teeth and gingival data are shown. In addition, model data obtained by expanding gingiva is needed when a three-dimensional tooth model is generated using three-dimensional scan data for tooth correction or prosthetic treatment.

When the gingival region selected from the scan data is expanded and the edge of the initially selected gingival region is not smooth, the edge of the initially selected gingival region may look angled. Accordingly, the gingival region may not look natural when the gingival region is expanded. Therefore, a method of selecting the gingival region is needed to make the gingival region have a gentle curve without forming an angle when the selected gingival region is expanded.

### DISCLOSURE

### Technical Problem

An object of a disclosed embodiment is to provide an intraoral image processing method and a device that performs operations thereof, for determining an edge of a region selected from three-dimensional scan data to look natural when the selected region is expanded.

### Technical Solution

An intraoral image processing method according to an embodiment may include acquiring three-dimensional intraoral data by scanning the oral cavity including teeth, acquiring a tooth region including teeth from the three-dimensional intraoral data, acquiring a gingival region the edge of which is formed by a curve having a smaller curvature than the curvature of the edge of the tooth region, based on the tooth region, and displaying the tooth region and the gingival region as a final selection region.

According to an embodiment, the acquiring of the tooth region may include acquiring the tooth region by identifying scan data about teeth and scan data about gingiva included in the three-dimensional intraoral data.

According to an embodiment, the acquiring of the tooth region may include, when the teeth are separated into individual tooth regions in the three-dimensional intraoral data, acquiring the tooth region by combining the individual tooth regions into one region.

According to an embodiment, the acquiring of the gingival region may include acquiring a curve that is a certain distance away from the edge of the tooth region, and determining the edge of the gingival region using some of points included in the curve.

According to an embodiment, the intraoral image processing method may further include displaying an object for setting the certain distance, and receiving a user input for the object to set the certain distance.

According to an embodiment, the acquiring of the gingival region may include determining the edge of the gingival region such that the edge of the gingival region has a smaller curvature than the curvature of the curve.

According to an embodiment, the intraoral image processing method may further include receiving a user input for selecting a palate region including scan data about a palate in the three-dimensional intraoral data, and determining a first reference point and a second reference point for distinguishing between a lingual side of teeth and a buccal side of teeth among points included in the curve, wherein the acquiring of the gingival region may include determining the edge of the gingival region based on points corresponding to the buccal side of teeth among points included in the curve.

According to an embodiment, the intraoral image processing method may further include acquiring, based on a curve connecting the first reference point to the second reference point on the lingual side of teeth, a second palate region excluding a partial region of the palate region, and displaying the second palate region as the final selection region.

According to an embodiment, the intraoral image processing method may further include acquiring tooth model data corresponding to the final selection region by generating mesh data extending from the edge of the final selection region.

According to an embodiment, an intraoral image processing device may include a display, memory storing one or more instructions, and a processor, wherein the processor is configured, by executing the one or more instructions stored in the memory, to acquire three-dimensional intraoral data by scanning the oral cavity including teeth, acquire a tooth region including the teeth from the three-dimensional intraoral data, acquire a gingival region the edge of which is formed by a curve having a smaller curvature than the curvature of the edge of the tooth region, based on the tooth region, and control the display to display the tooth region and the gingival region as a final selection region.

### Advantageous Effects

According to an intraoral image processing device and an intraoral image processing method according to a disclosed embodiment, an edge of a region selected from three-dimensional scan data may be determined to have a curve without forming an angle when the selected region is expanded. Accordingly, when the selected gingival region is expanded, it is possible to prevent the gingival region from looking unnatural.

### DESCRIPTION OF DRAWINGS

The present disclosure may be easily understood from a combination of the following detailed description with the accompanying drawings in which reference numerals refer to structural elements.

FIG. 1 is a diagram of an intraoral image processing system according to a disclosed embodiment.

FIG. 2 is a diagram of tooth model data combined with a base, according to an embodiment.

FIGS. 3 and 4 are diagrams for describing an operation, performed by an intraoral image processing device according to an embodiment, of selecting a region for generating tooth model data from scan data.

FIGS. 5A and 5B are diagrams for describing a method of adjusting a range of a gingival region selected by an intraoral image processing device according to an embodiment.

FIGS. 6A and 6B are diagrams for describing a method of selecting a region in scan data by using a selection tool, according to an embodiment.

FIGS. 7 to 8C are diagrams for describing an operation, performed by an intraoral image processing device according to an embodiment, of selecting a region including a palate region in scan data.

FIG. 9 is a flowchart of an intraoral image processing method according to an embodiment.

FIG. 10 is a block diagram of an intraoral image processing device according to an embodiment.

### BEST MODE

### Mode for Invention

The present specification clarifies the scope of rights of the present disclosure, explains the principles of the present disclosure, and discloses embodiments so that those skilled in the art can practice the present disclosure. The disclosed embodiments may be implemented in various forms.

Like reference numerals refer to like elements throughout the specification. The present specification does not describe all elements of the embodiments, and the general content or the overlapping content between the embodiments in the technical field to which the present disclosure belongs is omitted. The term “part” or “portion” used in the specification may be implemented as software or hardware, and according to embodiments, a plurality of “parts” or “portions” may be embodied as one unit or element, or one “part” or “portion” may include a plurality of units or elements. Hereinafter, the operating principles and embodiments of the present disclosure are described with reference to the attached drawings.

An image herein may include an image showing at least one tooth or an oral cavity including at least one tooth (hereinafter, referred to as an “intraoral image”).

In addition, the image herein may be a two-dimensional image of an object or a three-dimensional model or a three-dimensional image that represents the object three-dimensionally. In addition, the image herein may refer to data required to express the object in two or three dimensions, e.g., raw data acquired from at least one image sensor. Specifically, the raw data which is data acquired to generate an intraoral image may be data (e.g., two-dimensional data) acquired from at least one image sensor included in a three-dimensional scanner when scanning the oral cavity of a patient who is an object by using the three-dimensional scanner.

The “object” herein may include teeth, gingiva, at least a partial region of the oral cavity, and/or an artificial structure (e.g., orthodontic appliances, implants, artificial teeth, orthodontic aids inserted into the oral cavity, etc.) that can be inserted into the oral cavity. Here, the orthodontic appliances may include at least one of brackets, attachments, orthodontic screws, lingual orthodontic appliances, and removable orthodontic retainers.

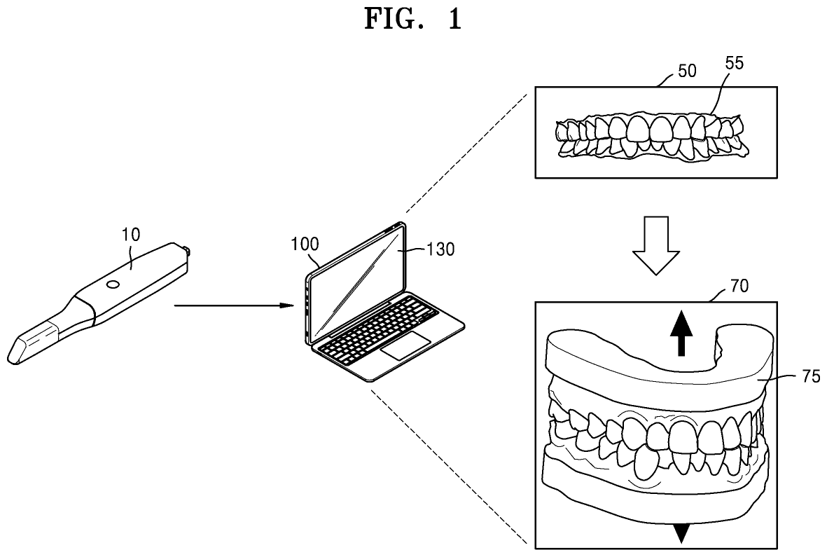

Hereinafter, embodiments are described in detail with reference to the drawings. FIG. 1 is a diagram of an intraoral image processing system according to a disclosed embodiment.

Referring to FIG. 1, the intraoral image processing system includes a three-dimensional scanner 10 and an intraoral image processing device 100.

The three-dimensional scanner 10 according to an embodiment is a device for scanning an object, and is a medical device for acquiring an image of an oral cavity. In addition, the three-dimensional scanner 10 may scan at least a portion of the body such as a patient's face, or a tooth model, in addition to the oral cavity.

The three-dimensional scanner 10 is illustrated in FIG. 1 in the form of a handheld scanner that is held by a user to scan an object, but is not limited thereto, and the three-dimensional scanner 10 may include a model scanner that scans a moving tooth model when the tooth model is installed.

Specifically, the three-dimensional scanner 10 may be a device that is inserted into the oral cavity and scans teeth in a non-contact manner to acquire an image of the oral cavity including at least one tooth. In addition, the three-dimensional scanner 10 may be capable of entering and exiting the oral cavity, and may scan the inside of the patient's oral cavity using at least one image sensor (e.g., an optical camera). The three-dimensional scanner 10 may acquire surface information about the object as raw data to image the surface of at least one of teeth, gingiva, and artificial structures (e.g., orthodontic appliances including brackets and wires, implants, artificial teeth, orthodontic aids inserted into the oral cavity, etc.) that can be inserted into the oral cavity, which is the object.

The image data acquired from the three-dimensional scanner 10 may be transmitted to the intraoral image processing device 100 connected thereto through a wired or wireless communication network.

The intraoral image processing device 100 connected to the three-dimensional scanner 10 through the wired or wireless communication network may be any electronic device capable of receiving a two-dimensional image acquired by scanning the oral cavity from the three-dimensional scanner 10, and generating, processing, displaying, and/or transmitting an intraoral image based on the received two-dimensional image.

The intraoral image processing device 100 may process the two-dimensional image data to generate information based on the two-dimensional image data received from the three-dimensional scanner 10, or process the two-dimensional image data to generate the intraoral image. In addition, the intraoral image processing device 100 may display the generated information and intraoral image through a display 130.

The intraoral image processing device 100 may be a computing device such as a smart phone, a laptop computer, a desktop computer, a personal digital assistant (PDA), or a tablet PC, but is not limited thereto.

In addition, the intraoral image processing device 100 may exist in the form of a server (or a server device) or the like for processing an intraoral image.

In addition, the three-dimensional scanner 10 may transmit raw data acquired through intraoral scanning to the intraoral image processing device 100 or partially process the raw data to be transmitted thereto. In this case, the intraoral image processing device 100 may generate a three-dimensional intraoral image three-dimensionally representing the oral cavity based on the received raw data. The intraoral image processing device 100 according to an embodiment may generate three-dimensional data (e.g., surface data, mesh data, etc.) three-dimensionally representing the shape of the surface of the object based on the received raw data.

In addition, since the “three-dimensional intraoral image” may be generated by three-dimensionally modeling the internal structure of the oral cavity based on the received raw data, it may also be referred to as a “three-dimensional intraoral model.” Hereinafter, a model or an image two-dimensionally or three-dimensionally representing the oral cavity is collectively referred to as an “intraoral image”.

In addition, the intraoral image processing device 100 may analyze, process, display, and/or transmit the generated intraoral image to an external device.

As another example, the three-dimensional scanner 10 may acquire raw data through intraoral scanning, process the acquired raw data, generate an image corresponding to the oral cavity that is an object, and transmit the image to the intraoral image processing device 100. In this case, the intraoral image processing device 100 may analyze, process, display, and/or transmit the received image.

In a disclosed embodiment, the intraoral image processing device 100 is an electronic device capable of generating and displaying an intraoral image three-dimensionally representing an oral cavity including one or more teeth, to be described in detail below.

When receiving raw data acquired by scanning the oral cavity from the three-dimensional scanner 10, the intraoral image processing device 100 according to an embodiment may process the received raw data to generate a three-dimensional intraoral image (or a three-dimensional intraoral model). For convenience of description, the three-dimensional intraoral image generated by the intraoral image processing device 100 is referred to as “scan data” below.

The raw data received from the three-dimensional scanner 10 may include tooth data representing teeth and gingival data representing gingiva. Accordingly, the scan data generated by the intraoral image processing device 100 may include a tooth region and a gingival region. Alternatively, the raw data according to an embodiment may include data representing a palate, and thus, the scan data may include a palate region. However, the present disclosure is not limited thereto.

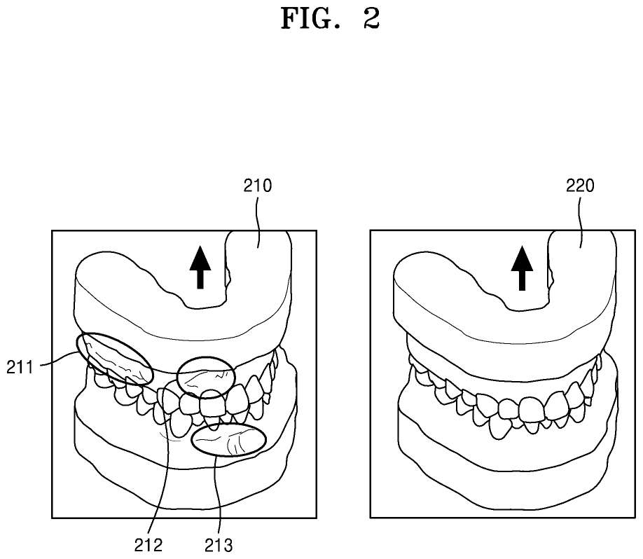

According to an embodiment, when a tooth model is generated using a tooth region and a portion of a gingival region 50 included in the scan data, an upper jaw and a lower jaw are separated, making it impossible to determine the correct occlusion of the teeth. Accordingly, it is necessary to generate tooth model data 70 combined with a base 75 to generate the tooth model with an attachable articulation for combining the upper jaw with the lower jaw. An edge of the gingival region 55 selected from the scan data needs to be expanded to the base 75 to generate the tooth model data 70 combined with the base 75. Referring to FIG. 2, a method of generating tooth model data by expanding the edge of the gingival region to the base is described.

FIG. 2 is a diagram of tooth model data combined with a base, according to an embodiment.

The intraoral image processing device 100 according to an embodiment may generate tooth model data combined with a base by using a tooth region and a portion of a gingival region, each selected from the scan data. The intraoral image processing device 100 may expand the gingival region to the base by generating three-dimensional data (e.g., mesh data) between the edge of the selected gingival region and the base.

In this case, when the edge of the gingival region selected from the scan data is not smooth, an angled or recessed shape 211, 212, and 213 may be generated in the expanded region included in the generated tooth model data 210. Accordingly, the generated tooth model data 210 may be in an unnatural form.

On the other hand, when the edge of the gingival region selected from the scan data is smooth, the expanded region included in the generated tooth model data 220 may have a gentle curve. Accordingly, the generated tooth model data 220 may be in a natural form.

Accordingly, when selecting the tooth region and a portion of the gingival region from the scan data to generate tooth model data, it is necessary to select the gingival region to make the edge of the gingival region look smooth.

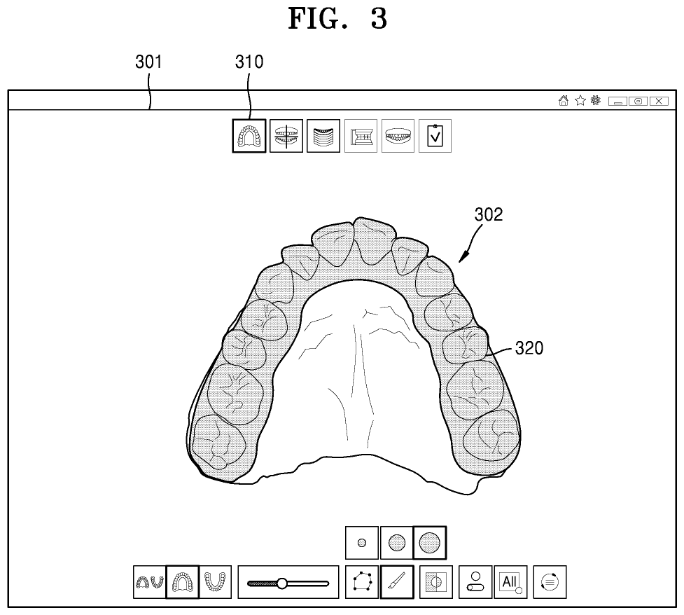

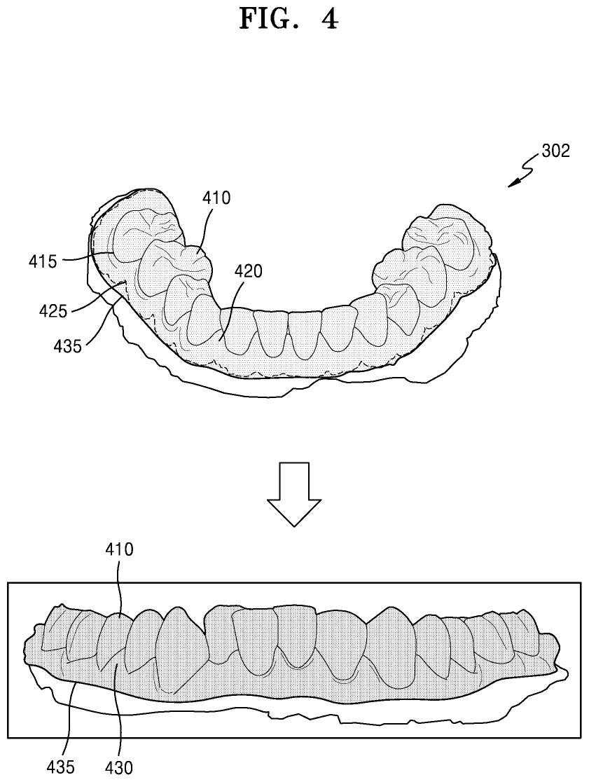

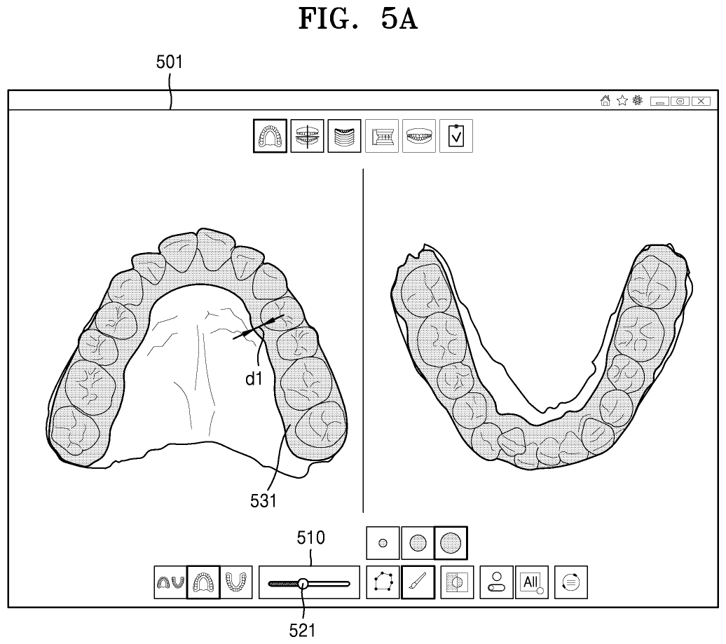

FIGS. 3 and 4 are diagrams for describing an operation of selecting a region for generating tooth model data from scan data, performed by an intraoral image processing device according to an embodiment.

The intraoral image processing device 100 according to an embodiment may generate scan data based on raw data acquired by the three-dimensional scanner 10. In addition, the intraoral image processing device 100 may visually output scan data 302 through a user interface screen 301. The user interface screen 301 may be a screen of the display 130 of FIG. 1. The user interface screen 301 may include at least one menu for allowing a user to analyze or process the scan data 302.

For example, the user interface screen 301 may include a region selection icon 310 for selecting a region in the scan data 302 to generate tooth model data.

When receiving a user input for selecting the region selection icon 310, the intraoral image processing device 100 may automatically select and display a region 320 for generating the tooth model data from the scan data 302. In this case, the selected region 320 may be displayed to be distinguished from the unselected region. For example, the selected region 320 may be highlighted in a different color from the unselected region, or the border of the selected region 320 may be displayed to be distinguished from the unselected region. However, the present disclosure is not limited thereto.

When receiving the user input for selecting the region selection icon 310 of FIG. 3, the intraoral image processing device 100 according to an embodiment may acquire a tooth region including teeth from the scan data 302.

Referring to FIG. 4, the intraoral image processing device 100 may acquire a tooth region 410 by segmenting the scan data 302 into a tooth region and a gingival region. Segmenting the scan data 302 into the tooth region and the gingival region may refer to separating tooth region in the scan data from the gingival region. The intraoral image processing device 100 may segment the scan data into the tooth region and the gingival region by identifying scan data about teeth and scan data about gingiva included in the scan data.

The intraoral image processing device 100 according to an embodiment may segment the scan data 302 into the tooth region and the gingival region by using artificial intelligence (AI) without a user input.

When teeth included in the tooth region are separated into individual tooth regions through segmentation, the intraoral image processing device 100 may combine the individual tooth regions into one region to acquire the tooth region 410.

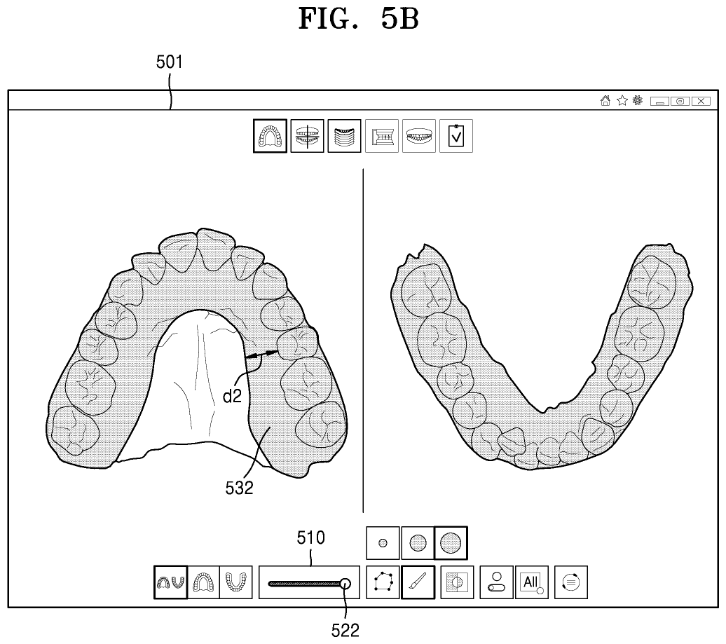

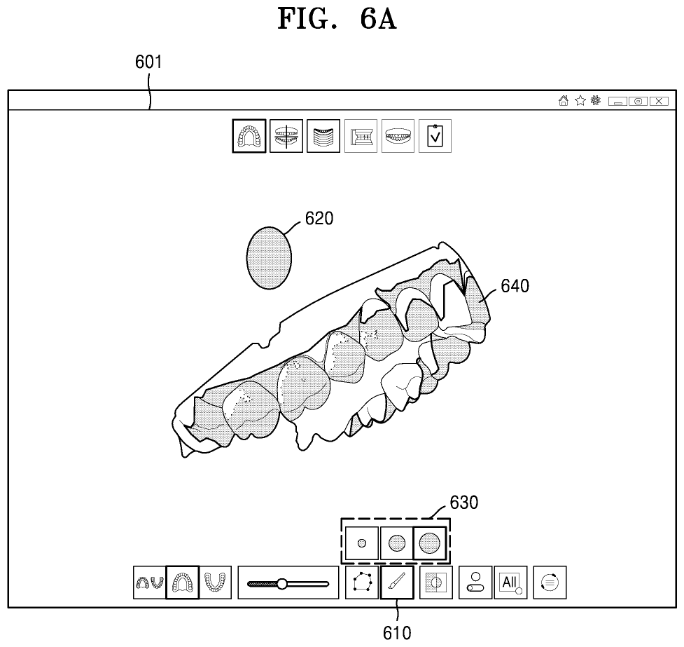

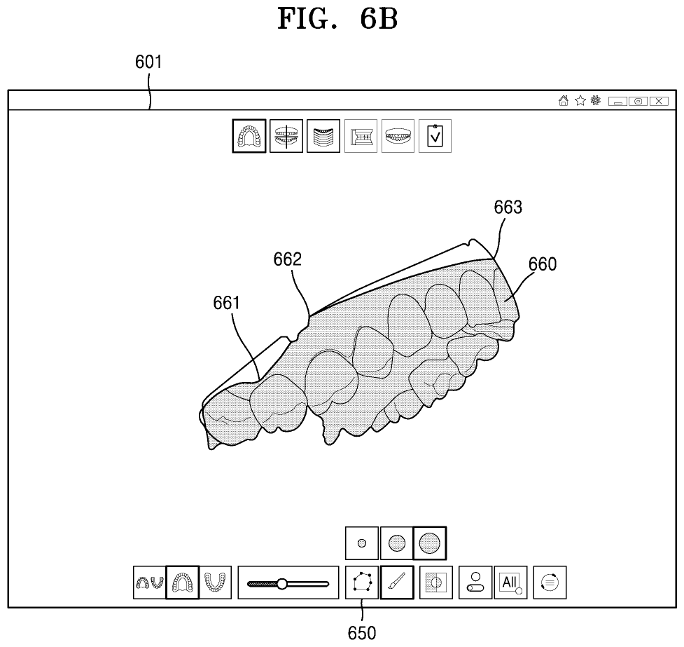

When the tooth region 410 is acquired, the intraoral image processing device 100 may acquire a first gingival region 420 the edge of which is formed by a first curve 425 that is a certain distance away from the edge of the tooth region 410. For example, the intraoral image processing device 100 may acquire the first curve 425 connecting vertices that are a certain distance away from vertices included in the edge 415 of the tooth region 410, respectively. In this case, the certain distance may be adjusted based on the user input. This is described below with reference to FIGS. 5A to 6.

When the first curve 425 is acquired, the intraoral image processing device 100 may acquire a region between the edge 415 of the tooth region 410 and the first curve 425 as the first gingival region 420.

The intraoral image processing device 100 may generate a second curve 435 by using some of points included in the first curve 425. The second curve 435 may be an interpolation curve that connects some of the points included in the first curve 425 so that the curvature of the second curve 435 is smaller than that of the first curve 425.

Alternatively, the points included in the first curve 425 may be grouped based on the x-axis coordinate value, and a point with the smallest z-axis coordinate value among the points included in one group may be selected for each group, and the selected points may be connected to generate the second curve 435. The intraoral image processing device 100 may acquire a region between the edge 415 of the tooth region 410 and the second curve 435 as a second gingival region 430.

Alternatively, the intraoral image processing device 100 may smooth the second curve 435 to acquire a region between the edge 415 of the tooth region 410 and the smoothed second curve as the second gingival region 430.

As shown in FIG. 3, the intraoral image processing device 100 may display the tooth region and the second gingival region as the final selection region 320.

Accordingly, the edge of the gingival region included in the final selection region 320 may have a gentle curve.

FIGS. 5A and 5B are diagrams for describing a method of adjusting a range of a gingival region selected by an intraoral image processing device according to an embodiment.

Referring to FIG. 5A, a user interface screen 501 may include an object 510 capable of adjusting a distance from the edge of the tooth region to the edge of a first gingival region (or a second gingival region). For example, the object 510 may include a scroll bar capable of moving the scroll to the left and right. When the scroll is moved to the left, the distance from the edge of the tooth region to the edge of the first gingival region (or the second gingival region) may decrease, and when the scroll moves to the right, the distances from the edge of the tooth region to the edge of the first gingival region (or the second gingival region) may increase.

As shown in FIG. 5A, when the scroll is located at a first point 521, the intraoral image processing device 100 may acquire a first gingival region the edge of which is formed by a curve that is a first distance d1 away from the edge of the tooth region, and may acquire a second gingival region 531 the edge of which is formed by a curve acquired by interpolating the edge of the first gingival region.

Meanwhile, as shown in FIG. 5B, when the scroll is located at a second point 522 to the right of the first point 521, the intraoral image processing device 100 may acquire a first gingival region the edge of which is formed by a curve that is a second distance d2 greater than the first distance d1 from the edge of the tooth region, and may acquire a second gingival region 532 the edge of which is formed by a curve acquired by interpolating the edge of the first gingival region. Accordingly, the second gingival region 532 of FIG. 5B is larger than the second gingival region 531 of FIG. 5A.

Accordingly, the user may easily adjust the range of the gingival region by using the scroll bar.

FIGS. 6A and 6B are diagrams for describing a method of selecting a region in scan data by using a selection tool, according to an embodiment.

As described in FIGS. 3 to 5B, the intraoral image processing device 100 according to an embodiment may automatically select the tooth region and a portion of the gingival region, but is not limited thereto, and may select a partial region from the scan data based on a user input. Alternatively, the intraoral image processing device 100 may automatically select the partial region based on the user input after the tooth region and a portion of the gingival region are selected.

The user interface screen 601 according to an embodiment may include at least one selection tool menu for selecting the partial region from the scan data.

For example, referring to FIG. 6A, the selection tool menu may include a brush menu 610. When a user input for selecting the brush menu 610 is received, the intraoral image processing device 100 may display a brush icon 620 and items 630 for selecting a size of the brush. When any one of the items 630 is selected, the intraoral image processing device 100 may change the size of the brush icon 620 to the selected size. The intraoral image processing device 100 may select a partial region 640 based on the user input for moving the brush icon 620.

In addition, referring to FIG. 6B, the selection tool menu may include a polygon selection menu 650. When the polygon selection menu 650 is selected and a user input for selecting at least one point 661, 662, or 663 included in the scan data is received, the intraoral image processing device 100 may connect the selected points 661, 662 and 633 to generate a polygon, and may select an inner region 660 of the polygon.

When selecting a partial region from the scan data based on a user input using a selection tool, the intraoral image processing device 100 according to an embodiment may generate a curve to make the edge of the selected region gentle, and may change the generated curve to the edge of the selected region.

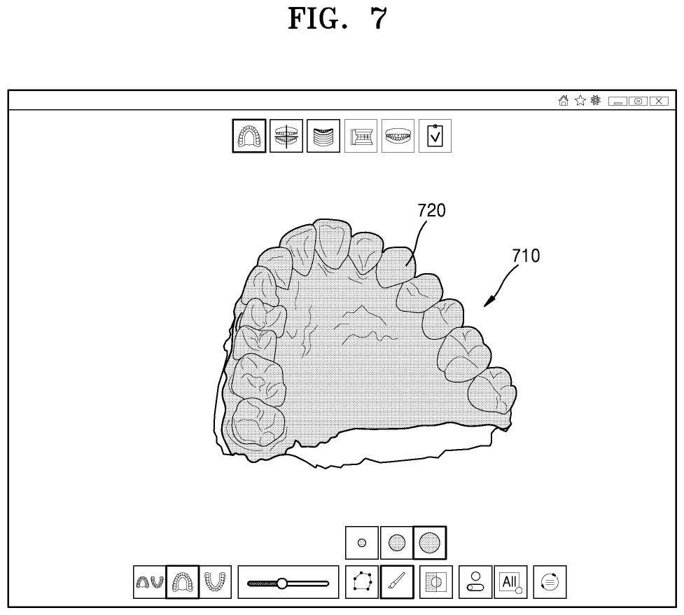

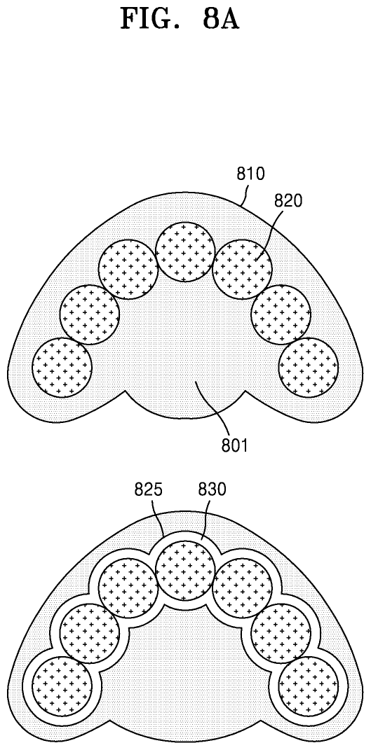

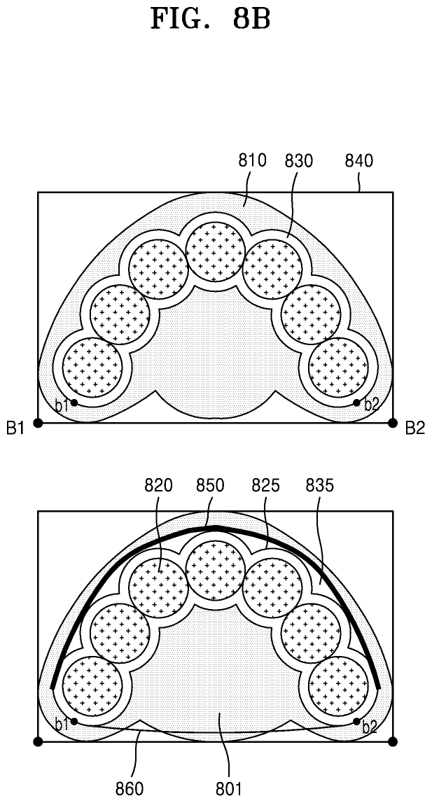

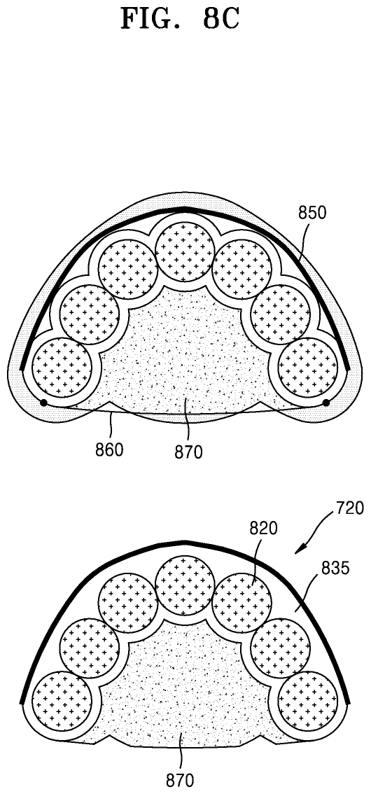

FIGS. 7 to 8C are diagrams for describing an operation of selecting a region including a palate region in scan data, performed by an intraoral image processing device according to an embodiment.

Referring to FIG. 7, scan data 710 according to an embodiment may include scan data about a palate. The intraoral image processing device 100 may select a region to include a palate region from the scan data 710. In this case, the user input may determine whether to include the palate region. For example, the user may set the tooth model data including the palate region to be generated through a menu or the like, or may further select the palate region by using the selection tool such as a brush or a polygon. However, the present disclosure is not limited thereto.

As shown in FIG. 7, the intraoral image processing device 100 according to an embodiment may select and display a partial region 720 of the scan data 710 to include the palate region. An operation of selecting a region including the palate region from the scan data, performed by the intraoral image processing device 100 according to an embodiment is described in detail with reference to FIGS. 8A to 8C.

Referring to FIG. 8A, scan data 810 according to an embodiment may include a palate region 801. The intraoral image processing device 100 may acquire a tooth region 820 from the scan data 810. In this case, since a method of acquiring the tooth region 820 from the scan data 810 has been described in detail with reference to FIG. 4, a detailed description thereof is omitted.

When the tooth region 820 is acquired, the intraoral image processing device 100 may acquire a first gingival region 830 the edge of which is formed by a first curve 825 that is a certain distance away from the edge of the tooth region 820. Since a method of acquiring the first gingival region 830 is also described in detail with reference to FIG. 4, a detailed description thereof is omitted.

When the first gingival region 830 is acquired, the intraoral image processing device 100 may determine a first reference point and a second reference point for distinguishing between a buccal side of teeth and a lingual side of teeth among points included in the edge of the first gingival region 830.

For example, as shown in FIG. 8B, the intraoral image processing device 100 may acquire a bounding box 840 including the scan data 810. In this case, the scan data 810 may be aligned so that the buccal direction of teeth is positioned in a first direction (e.g., upward) and the lingual direction of teeth is positioned in a second direction (e.g., downward).

The intraoral image processing device 100 may determine two points closest to lower two vertices B1 and B2 of the bounding box 840, respectively, among the points included in the edge of the first gingival region 830 as a first reference point b1 and a second reference point b2.

The intraoral image processing device 100 may generate a second curve 850 based on some of the points included in a portion of the edge 825 of the first gingival region 830 corresponding to the buccal side of teeth between the first reference point b1 and the second reference point b2. The intraoral image processing device 100 may acquire a region from the edge of the tooth region 820 to the second curve 850 as the second gingival region 835.

In addition, the intraoral image processing device 100 may generate a third curve 860 connecting the first reference point b1 to the second reference point b2 on the lingual side of teeth, and may acquire a second palate region 870 excluding a partial region of the palate region 801 based on the third curve 860.

For example, as shown in FIG. 8C, the data of a portion of the palate region 801 located outside the third curve 860 based on the third curve 860 may be excluded from a final selection region.

As shown in FIG. 8C, the intraoral image processing device 100 may display the tooth region 820, the second gingival region 835, and the second palate region 870 as the final selection region. As a result, the edge of the final selection region may have a gentle curve.

The intraoral image processing device 100 according to an embodiment may generate tooth model data based on the final selection region. For example, the intraoral image processing device 100 may expand the gingival region to the base by generating mesh data between the base determined based on the final selection region and the edge of the final selection region.

In this case, since the edge of the final selection region has a gentle curve shape, the expanded region may have a gently curved shape.

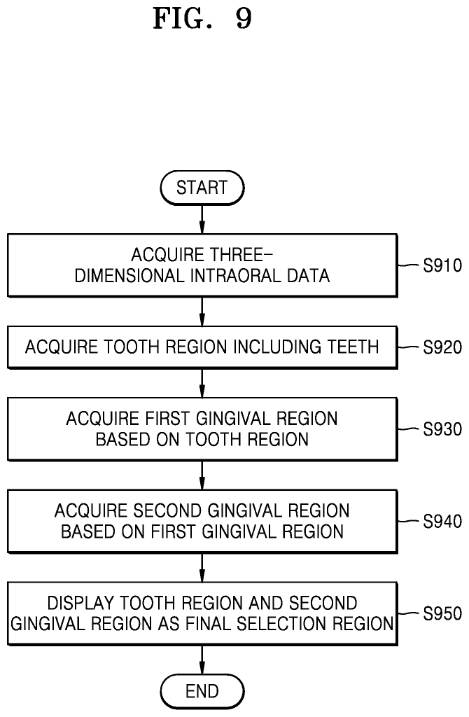

FIG. 9 is a flowchart of an intraoral image processing method according to an embodiment.

The intraoral image processing method illustrated in FIG. 9 may be performed by the intraoral image processing device 100.

Referring to FIG. 9, the intraoral image processing device 100 according to an embodiment may acquire three-dimensional intraoral data (scan data) (S910).

The intraoral image processing device 100 may receive raw data acquired by scanning an oral cavity including teeth or scanning a tooth model using the three-dimensional scanner 10, and may generate the scan data based on the received raw data. Alternatively, the intraoral image processing device 100 may acquire the scan data stored in memory.

The intraoral image processing device 100 may acquire a tooth region including teeth from the scan data (S920).

For example, the intraoral image processing device 100 may acquire the tooth region by segmenting the scan data into a tooth region and a gingival region. Segmenting the scan data into the tooth region and the gingival region may refer to separating teeth from the gingival region in the scan data. The intraoral image processing device 100 may segment the scan data into the tooth region and the gingival region by identifying scan data about teeth and scan data about gingiva included in the scan data.

The intraoral image processing device 100 according to an embodiment may acquire a first gingival region based on the tooth region (S930).

When the tooth region is acquired, the intraoral image processing device 100 may acquire a first gingival region the edge of which is formed by a first curve that is a certain distance away from the edge of the tooth region. For example, the intraoral image processing device 100 may acquire a first curve connecting vertices that are a certain distance away from the vertices included in the edge of the tooth region, respectively. In this case, the certain distance may be adjusted based on a user input.

The intraoral image processing device 100 according to an embodiment may acquire a second gingival region based on the first gingival region (S940).

For example, the intraoral image processing device 100 may generate a second curve by using some of points included in the edge (first curve) of the first gingival region. In this case, the second curve may be a curve connecting some of the points included in the first curve so that the curvature of the second curve is smaller than that of the first curve. The second curve has a gentler shape than the first curve.

The intraoral image processing device 100 may acquire a region between the edge of the tooth region and the second curve as the second gingival region.

The intraoral image processing device 100 according to an embodiment may display the tooth region and the second gingival region as a final selection region (S950).

Accordingly, the edge of the gingival region included in the final selection region may be formed with a gentle curve.

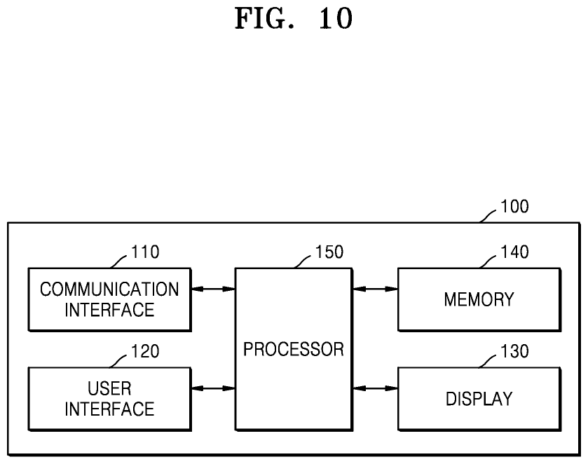

FIG. 10 is a block diagram of an intraoral image processing device according to an embodiment.

The intraoral image processing method illustrated in FIG. 9 may be performed by the intraoral image processing device 100. Accordingly, the intraoral image processing method illustrated in FIG. 9 may be a flowchart illustrating operations performed by the intraoral image processing device 100.

Referring to FIG. 10, the intraoral image processing device 100 may include a communication interface 110, a user interface 120, a display 130, memory 140, and a processor 150.

The communication interface 110 may perform communication with at least one external electronic device (e.g., three-dimensional scanner 10, server, or external medical device) through a wired or wireless communication network. The communication interface 110 may perform communication with at least one external electronic device, controlled by the processor 150.

Specifically, the communication interface 110 may include at least one short-range communication module that performs communication according to a communication standard such as Bluetooth, WiFi, Bluetooth low energy (BLE), NFC/RFID, WiFi Direct, UWB, or ZIGBEE.

In addition, the communication interface 110 may further include a long-distance communication module that performs communication with a server for supporting long-distance communication according to a long-distance communication standard. Specifically, the communication interface 110 may include a long-distance communication module that performs communication through a network for Internet communication. In addition, the communication interface 110 may include a long-distance communication module that performs communication through a communication network according to a communication standard such as 3G, 4G, and/or 5G.

In addition, the communication interface 110 may include at least one port for connection with an external electronic device (e.g., three-dimensional scanner, etc.) via a wired cable, to perform communication with the external electronic device. Accordingly, the communication interface 110 may perform communication with the external electronic device wired thereto through at least one port.

The user interface 120 may receive a user input for controlling the intraoral image processing device 100. The user interface 120 may include, but is not limited to, a user input device including a touch panel that detects a user's touch, a button that receives push operations from the user, a mouse or a keyboard for designating or selecting a point on the user interface screen, and the like.

In addition, the user interface 120 may include a voice recognition device for voice recognition. For example, the voice recognition device may be a microphone, and the voice recognition device may receive a voice command or a voice request from the user. Accordingly, the processor 150 may control an operation corresponding to the voice command or the voice request to be performed.

The display 130 displays a screen. Specifically, the display 130 may display a certain screen controlled by the processor 150. Specifically, the display 130 may display a user interface screen including an intraoral image generated based on data acquired by scanning the patient's oral cavity with the three-dimensional scanner 10. Alternatively, the display 130 may display a user interface screen including information related to dental treatment of the patient.

The memory 140 may store at least one instruction. In addition, the memory 140 may store at least one instruction executed by the processor 150. In addition, the memory 140 may store at least one program executed by the processor 150. In addition, the memory 140 may store data received from the three-dimensional scanner 10 (e.g., raw data acquired through intraoral scanning, etc.). Alternatively, the memory 140 may store an intraoral image three-dimensionally representing the oral cavity. The memory 140 according to an embodiment may include one or more instructions for deleting noise data from the intraoral image. The memory 140 according to an embodiment may include one or more instructions for performing the method disclosed in the present disclosure to delete noise data from the intraoral image.

The processor 150 performs at least one instruction stored in the memory 140 to control an intended operation to be performed. The at least one instruction herein may be stored in internal memory included in the processor 150 or in the memory 140 included in a data processing device separately from the processor 150.

Specifically, the processor 150 may perform at least one instruction to control at least one component included in the data processing device so that the intended operation is performed. Therefore, although the case where the processor performs certain operations is illustrated herein, it may indicate that the processor controls at least one component included in the data processing device so that certain operations are performed.

The processor 150 according to an embodiment may generate scan data based on raw data acquired by executing one or more instructions stored in the memory 140, scanning the oral cavity including teeth, or scanning the tooth model. The processor 150 may acquire the tooth region including teeth from the scan data by executing one or more instructions stored in the memory 140. The processor 150 may acquire the tooth region by segmenting the scan data into the tooth region and the gingival region.

The processor 150 may acquire a first gingival region based on the tooth region by executing one or more instructions stored in the memory 140. When the tooth region is acquired, the processor 150 may acquire the first gingival region the edge of which is formed by a first curve that is a predetermined distance away from the edge of the tooth region. For example, the processor 150 may acquire a first curve connecting vertices that are a certain distance away from the vertices included in the edge of the tooth region, respectively. The processor 150 may acquire a second gingival region based on the first gingival region by executing one or more instructions stored in the memory 140. The processor 150 may generate a second curve by using some of the points included in the edge (first curve) of the first gingival region. In this case, the second curve may be a curve connecting some of the points included in the first curve so that the curvature of the second curve is smaller than that of the first curve. The second curve includes a gentler shape than the first curve.

The processor 150 may acquire a region between the edge of the tooth region and the second curve as the second gingival region.

The processor 150 may display the tooth region and the second gingival region as the final selection region.

By executing one or more instructions stored in the memory 140, the processor 150 may determine a first reference point and a second reference point for distinguishing between the buccal side of teeth and the lingual side of teeth when selecting a region including the palate region in the scan data. The processor 150 may generate a curve based on some of the points included in a portion of the edge of the first gingival region corresponding to the buccal side of teeth between the first reference point and the second reference point, and may acquire the second gingival region based on the generated curve.

In addition, the processor 150 may determine a second palate region based on a curve connecting the first reference point and the second reference point on the lingual side of the tooth, may generate a curve based on some of the points included in the edge of the second palate region, and may acquire a third palate region the edge of which is formed by the generated curve.

The processor 150 may display the tooth region, the second gingival region, and the third palate region as the final selection region.

The processor 150 according to an embodiment may be implemented as including at least one internal processor and a memory device (e.g., RAM, ROM, etc.) for storing at least one of a program, an instruction, a signal, and data to be processed or used in the internal processor.

In addition, the processor 150 may include a graphic processing unit (GPU) for graphic processing corresponding to videos. In addition, the processor 150 may be implemented as a system on chip (SoC) in which a core and a GPU are integrated. In addition, the processor 150 may include more than a single core or multiple cores. For example, the processor 150 may include dual cores, triple cores, quad-cores, hexa-cores, octa-cores, deca-cores, dodeca-cores, hexa-dash-vale cores, and the like.

In a disclosed embodiment, the processor 150 may generate an intraoral image based on a two-dimensional image received from the three-dimensional scanner 10.

Specifically, according to the control of the processor 150, the communication interface 110 may receive data acquired from the three-dimensional scanner 10, for example, raw data acquired through intraoral scanning. In addition, the processor 150 may generate a three-dimensional intraoral image three-dimensionally representing the oral cavity based on the raw data received from the communication interface 110. For example, the three-dimensional scanner 10 may include an L-camera corresponding to a left field of view and an R-camera corresponding to a right field of view to restore the three-dimensional image according to an optical triangulation method. In addition, the three-dimensional scanner 10 may acquire L-image data corresponding to a left field of view and R-image data corresponding to a right field of view from the L-camera and the R-camera, respectively. Subsequently, the three-dimensional scanner 10 may transmit raw data including the L-image data and R-image data to the communication interface 110 of the intraoral image processing device 100.

Then, the communication interface 110 may transmit the received raw data to the processor 150, and the processor 110 may generate an intraoral image three-dimensionally representing the oral cavity based on the transmitted raw data.

In addition, the processor 150 may control the communication interface 110 to directly receive the intraoral image three-dimensionally representing the oral cavity from an external server, a medical device, or the like. In this case, the processor 150 may not generate the three-dimensional intraoral image based on the raw data, but may acquire the three-dimensional intraoral image.

According to a disclosed embodiment, that the processor 150 performs operations such as “extraction”, “acquisition”, or “generation” may include not only a case where the processor 150 directly performs the aforementioned operations by executing at least one instruction but also a case where the processor 150 controls other components to perform the aforementioned operations.

To implement the embodiments disclosed in the present disclosure, the intraoral image processing device 100 may include only some of the components shown in FIG. 10, or may include more components than those shown in the FIG. 10.

In addition, the intraoral image processing device 100 may store and execute dedicated software linked to three-dimensional scanners. The dedicated software herein may be referred to as a dedicated program, a dedicated tool, or a dedicated application. When the intraoral image processing device 100 operates in cooperation with the three-dimensional scanner 10, the dedicated software stored in the intraoral image processing device 100 may be connected with the three-dimensional scanner 10 to receive data acquired through intraoral scanning in real time. For example, dedicated software to process data obtained through intraoral scanning is in Medit's three-dimensional scanner, i500. Specifically, Medit produces and distributes “Medit Link”, software for processing, managing, using, and/or transmitting data acquired from three-dimensional scanners (e.g., i500). The “dedicated software” herein refers to a program, a tool, or an application that operates in cooperation with three-dimensional scanners, and thus may be commonly used by various three-dimensional scanners developed and sold by various manufacturers. In addition, the aforementioned dedicated software may be produced and distributed separately from the three-dimensional scanner that performs intraoral scanning.

The intraoral image processing device 100 may store and execute the dedicated software corresponding to i500. The dedicated software may perform at least one operation for acquiring, processing, storing, and/or transmitting the intraoral image. The dedicated software herein may be stored in a processor. In addition, the dedicated software may provide a user interface for the use of data acquired from three-dimensional scanners. The user interface screen provided in the dedicated software may include an intraoral image generated according to a disclosed embodiment.

The intraoral image processing method according to an embodiment may be implemented in the form of program instructions that can be executed through various computer means, and may be recorded on a computer-readable medium. In addition, an embodiment may be a computer-readable storage medium having one or more programs recorded thereon, including at least one instruction for executing the intraoral image processing method.

The computer-readable storage medium may include program instructions, data files, data structures, and the like, alone or in combination. Examples of the computer-readable storage medium herein may include magnetic media such as a hard disk, a floppy disk, and a magnetic tape, optical media such a CD-ROM and a DVD, magneto-optical media such an optical disk, and hardware devices configured to store and perform program instructions such as ROM, RAM, and flash memory.

A device-readable storage medium may be provided in the form of a non-transitory storage medium. The “non-transitory storage medium” herein may refer to a tangible storage medium. In addition, the “non-transitory storage medium” may include a buffer in which data is temporarily stored.

According to an embodiment, an intraoral image processing method according to some embodiments may be included and provided in a computer program product. The computer program product may be distributed in the form of a device-readable storage medium (e.g., CD-ROM). Alternatively, the computer program product may be distributed online (e.g., downloading or uploading), through an application store (e.g., Play Store) or directly between two user devices (e.g., smart phones). Specifically, the computer program product according to a disclosed embodiment may include a storage medium on which a program including at least one instruction for performing the intraoral image processing method according to a disclosed embodiment is recorded.

Although the embodiments have been described in detail above, the scope of the present disclosure is not limited thereto, and various modifications and improvements of those skilled in the art using the basic concept of the present disclosure defined in the following claims also fall within the scope.

1. An intraoral image processing method comprising:
acquiring three-dimensional intraoral data by scanning an oral cavity including teeth;
acquiring a tooth region including teeth from the three-dimensional intraoral data;
acquiring a gingival region having an edge formed as a curve having a smaller curvature than a curvature of an edge of the tooth region, based on the tooth region; and
displaying the tooth region and the gingival region as a final selection region.

2. The intraoral image processing method of claim 1,
wherein the acquiring of the tooth region comprises
acquiring the tooth region by identifying scan data about teeth and scan data about gingiva included in the three-dimensional intraoral data.

3. The intraoral image processing method of claim 1,
wherein the acquiring of the tooth region comprises,
when the teeth are separated into individual tooth regions in the three-dimensional intraoral data, acquiring the tooth region by combining the individual tooth regions into one region.

4. The intraoral image processing method of claim 1,
wherein the acquiring of the gingival region comprises:
acquiring a curve that is a certain distance away from the edge of the tooth region; and
determining the edge of the gingival region using some of points on the curve.

5. The intraoral image processing method of claim 4,
wherein the intraoral image processing method further comprises:
displaying an object for setting the certain distance; and
receiving a user input for the object, and setting the certain distance.

6. The intraoral image processing method of claim 4,
wherein the acquiring of the gingival region comprises
determining the edge of the gingival region such that the edge of the gingival region has a smaller curvature than the curvature of the curve.

7. The intraoral image processing method of claim 4,
wherein the intraoral image processing method further comprises:
receiving a user input for selecting a palate region including scan data about a palate in the three-dimensional intraoral data; and
determining a first reference point and a second reference point for distinguishing between a lingual side of teeth and a buccal side of teeth among points on the curve;
wherein the acquiring of the gingival region comprises
determining the edge of the gingival region based on points corresponding to the buccal side of teeth among points on the curve.

8. The intraoral image processing method of claim 7,
wherein the intraoral image processing method further comprises:
acquiring, based on a curve connecting the first reference point to the second reference point on the lingual side of teeth, a second palate region excluding a partial region of the palate region; and
displaying the second palate region as the final selection region.

9. The intraoral image processing method of claim 1,
wherein the intraoral image processing method further comprises
acquiring tooth model data corresponding to the final selection region by generating mesh data extending from the edge of the final selection region.

10. An intraoral image processing device comprising:
a display;
memory storing one or more instructions; and
a processor,
wherein the processor is configured, by executing the one or more instructions stored in the memory, to:
acquire three-dimensional intraoral data by scanning an oral cavity including teeth;
acquire a tooth region including the teeth from the three-dimensional intraoral data;
acquire a gingival region having an edge formed as a curve with a smaller curvature than a curvature of an edge of the tooth region, based on the tooth region, and
control the display to display the tooth region and the gingival region as a final selection region.

11. The intraoral image processing device of claim 10,
wherein the processor is configured, by executing the one or more instructions stored in the memory, to
acquire the tooth region by identifying scan data about teeth and scan data about gingiva included in the three-dimensional intraoral data.

12. The intraoral image processing device of claim 10,
wherein the processor is configured, by executing the one or more instructions stored in the memory, to:
when the teeth are separated into individual tooth regions in the three-dimensional intraoral data, acquire the tooth region by combining the individual tooth regions into one region.

13. The intraoral image processing device of claim 10,
wherein the processor is configured, by executing the one or more instructions stored in the memory, to:
acquire a curve that is a certain distance away from the edge of the tooth region, and
acquire the gingival region by determining the edge of the gingival region by using some of points on the curve.

14. The intraoral image processing device of claim 13,
wherein the processor is configured, by executing the one or more instructions stored in the memory, to:
control the display to display an object for setting the certain distance, receive a user input for the object, and set the certain distance.

15. The intraoral image processing device of claim 10,
wherein the processor is configured, by executing the one or more instructions stored in the memory, to:
determine the edge of the gingival region such that the edge of the gingival region has a smaller curvature than the curvature of the curve.

16. The intraoral image processing device of claim 13,
wherein the device further comprises
a user interface for receiving a user input for selecting a palate region including scan data about a palate in the three-dimensional intraoral data,
wherein the processor is configured, by executing the one or more instructions stored in the memory, to:
determine a first reference point and a second reference point for distinguishing between a lingual side of teeth and a buccal side of tooth among points on the curve based on receiving the user input for selecting the palate region, and
determine the edge of the gingival region based on points corresponding to the buccal side of teeth among the points included in the curve.

17. The intraoral image processing device of claim 16,
wherein the processor is configured, by executing the one or more instructions stored in the memory, to:
acquire, based on a curve connecting the first reference point and the second reference point on the lingual side of teeth, a second palate region excluding a partial region of the palate region, and
control the display to display the second palate region as the final selection region.

18. The intraoral image processing device of claim 10,
wherein the processor is configured, by executing the one or more instructions stored in the memory, to
acquire tooth model data corresponding to the final selection region by generating mesh data extending from the edge of the final selection region.

19. A computer-readable recording medium on which a program including at least one instruction for performing an intraoral image processing method by a computer is recorded, the intraoral image processing method comprising:
acquiring three-dimensional intraoral data by scanning an oral cavity including teeth;
acquiring a tooth region including the teeth in the three-dimensional intraoral data;
acquiring a gingival region having an edge formed as a curve having a smaller curvature than a curvature of an edge of the tooth region, based on the tooth region; and
displaying the tooth region and the gingival region as a final selection region.
